(19) United States
(12) Patent Application Publication
(10) Pub. No.: US 2012/0094951 A1
(43) Pub. Date: Apr. 19, 2012

(54) COMPOSITIONS

(76) Inventor: MARK ROBINSON, PATTERSON LAKES (AU)

(21) Appl. No.: 13/273,590

(22) Filed: Oct. 14, 2011

Related U.S. Application Data
(60) Provisional application No. 61/393,530, filed on Oct. 15, 2010.

(51) Int. Cl. A61K 31/726; A61P 19/02

(52) U.S. Cl. 514/54

(57) ABSTRACT

The specification relates to compositions comprising honey and chondroitin and/or glucos amine wherein the ingredients form a stably homogeneous composition. The subject compositions are contemplated for oral use as nutraceuticals or functional food to provide health benefits to subjects in need.

### FIELD

The specification relates to compositions comprising honey and chondroitin and/or glucosamine wherein the ingredients form a stably homogeneous composition. The subject compositions are contemplated for oral use as nutraceuticals or functional food to provide health benefits to subjects in need.

### BACKGROUND

Bibliographic details of the publications referred to by author in this specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Honey is produced by bees which have been “farmed” by man since ancient times to provide honey for its sweetening and reputed health-giving properties. Honey is mainly a mixture of fructose and glucose with lower amounts of maltose and sucrose and other complex carbohydrates. Honey further comprises small amounts of vitamins, minerals and antioxidants. It also has a low pH and low water activity. Honey is generally a sticky viscous liquid substance which becomes more liquid in warm temperatures and becomes less liquid in cooler temperatures.

Glycosaminoglycans are naturally occurring polysaccharide macromolecules which are found in living organisms where they form proteoglycans that are abundant in the connective tissues of bony animals and fish. Chondroitin sulphate is a sulphated glycosaminoglycan comprising repeated disaccharide units of N-acetylgalactosamine and glucuronic acid. Most of the N-acetylgalactosamine residues are sulphated. Chondroitin is a highly hydrophilic and gel forming and its hydrocolloidal properties provide much of the compressive resistance of cartilage.

Chondroitin sulphate has been used to treat or prevent various inflammatory or degenerative conditions, however, its most widespread use is in the treatment of arthritis.

Glucos amine is an amino sugar that is a building block for glycosaminoglycans that are part of cartilage. Glucosamine is also abundant in chitosan and chitin, which is found in the exoskeletons of crustaceans and other arthropods, the cell walls in fungi and in many higher organisms. Glucosamine is one of the most commonly used non-vitamin, non-mineral, dietary supplements.

Glucosamine, as glucosamine hydrochloride or glucosamine sulphate, and chondroitin sulphate either separately or combined are increasingly recommended, in guidelines promulgated by general practitioners and rheumatologists, for use in modifying inflammatory processes in osteoarthritis and in the relief of arthritis. These preparations are generally formulated in tablet or capsule form to provide maximum stability during processing and storage.

A major risk factor in the development of inflammatory conditions such as arthritis is advancing age. For the elderly, who may be confronted by a complex array of medications often requiring even more complex dosing schedules, compliance is an increasing problem and the more medications that can be taken as “food” rather than as tablets or capsules, the better. Reducing the amount of tablets or capsules that need to be taken is also important as swallowing tablets and capsules is often unpleasant and can contribute to a lack of compliance. Powdered forms of chondroitin sulphate and glucos amine are available that can be sprinkled on food, however, these powders are generally not homogeneous and are fairly unpalatable. There is a need for nutraceutical or functional food products that provide health benefits to subjects in need without adding to their tablet burden, thereby facilitating compliance.

### SUMMARY

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the singular forms “a”, “an” and “the” include plural aspects unless the context clearly dictates otherwise. Thus, for example, reference to “a composition” includes a single composition, as well as two or more compositions; reference to “an agent” includes one agent, as well as two or more agents; reference to “the composition” includes single and multiple embodiments of the composition, and so forth.

Each embodiment in this specification is to be applied mutatis mutandis to every other embodiment unless expressly stated otherwise.

In one broad embodiment, the present invention provides a substantially stably homogeneous composition comprising honey and chondroitin.

In some embodiments, the chondroitin is present in the composition at a concentration sufficient to provide a therapeutic dose in an acceptable amount of honey for ingestion.

In some embodiments, the composition comprises a final concentration of at least 16 mg to 100 mg chondroitin/g honey.

The compositions of the present invention are suitable for oral administration to a human subject with or without other food items.

In one broad embodiment, the present invention provides a substantially stably homogeneous composition comprising honey and glucosamine.

In some embodiments, the glucosamine is present in the composition at a concentration sufficient to provide a therapeutic dose in an acceptable amount of honey for ingestion.

In some embodiments, the composition comprises a final concentration of at least 25 mg to 150 mg glucosamine/g honey.

In some embodiments, the present invention provides a stably homogeneous composition comprising honey and chondroitin and glucosamine.

In a further embodiment, the chondroitin and glucos amine are present in the composition at concentrations sufficient to provide therapeutic doses in an acceptable amount of honey for ingestion. Therapeutic doses may vary depending upon the advice of the medical professional and lower doses are also expressly contemplated by the present invention.

In some embodiments, the composition comprises a final concentration of at least 16 mg to 100 mg chondroitin/g honey and at least 25 mg to 150 mg glucosamine/g honey.

In another aspect, the present invention provides a process for preparing a composition of honey comprising chondroitin in a substantially stably homogeneous form, the process comprising (i) obtaining honey.

In some embodiments, step (i) comprises heating the honey to at least about 40° C. to 65° C.

In some embodiments, the process further comprises (ii) contacting honey with an amount of chondroitin for a time and under conditions sufficient to form a final concentration that provides a therapeutic dose in an acceptable amount of honey for ingestion on a semi-regular, regular or daily basis.

In some embodiments of the process, honey is contacted with chondroitin in an amount sufficient to form a final concentration in the range of 16 mg to 100 mg chondroitin/g honey.

In some embodiments, step (ii) comprises heating the honey and chondroitin to at least about 40° C. to 65° C.

In some embodiments, the process comprises (iii) blending liquid honey and chondroitin for at least 12 hours to at least 17 hours.

Importantly, after the blending step, compositions of the present invention are stably homogeneous and fail to separate on standing.

In some embodiments, the present invention provides a process for preparing a composition comprising honey and chondroitin in a substantially stably homogeneous form, the process comprising (i) obtaining honey including optionally heating same to at least about 40° C. to 65° C.; (ii) contacting honey with a quantity of chondroitin for a time and under conditions sufficient to form a final concentration in the range of 16 mg to 100 mg chondroitin/g honey; (iii) blending liquid honey and chondroitin for at least 12 hours to at least 17 hours after which the composition is stably homogeneous and fails to separate on standing.

If the honey is not liquid it may be heated to liquid either before or after the addition of chondroitin or glucosamine or chondroitin and glucosamine.

In another aspect, the present invention provides a process for preparing a composition comprising honey and glucosamine in a substantially stably homogeneous form, the process comprising (i) obtaining honey.

In some embodiments, honey is heated to at least about 40° C. to 65° C.

In some embodiments, the process further comprises (ii) contacting honey with a quantity of glucosamine for a time and under conditions sufficient to form a final concentration in an amount sufficient to provide a therapeutic dose in an acceptable amount of honey for ingestion on a semi-regular, regular or daily basis.

In some embodiments of the process, honey is contacted with glucos amine in an amount sufficient to form a final concentration in the range of 25 mg to 150 mg glucosamine/g honey.

In some embodiments, honey and glucosamine are heated to at least about 40° C. to 65° C.

In some embodiments, the process further comprises (iii) blending liquid honey and glucosamine for at least 12 hours to at least 17 hours. After the blending step, the composition is stably homogeneous and fails to separate on standing.

In another aspect, the present invention provides a process for preparing a composition comprising honey and glucosamine in a substantially stably homogeneous form, the process comprising (i) obtaining honey including optionally heating same to at least about 40° C. to 65° C.; (ii) contacting honey with a quantity of glucosamine sufficient to form a final concentration in the range of 25 mg to 150 mg glucosamine/g honey; and (iii) blending liquid honey and glucosamine for at least 12 hours to at least 17 hours. After the blending step, the composition is stably homogeneous and fails to separate on standing.

In another aspect, the present invention provides a process for preparing a composition comprising honey comprising chondroitin and glucosamine in a substantially stably homogeneous form, the process comprising (i) obtaining honey.

In some embodiments, honey is heated to at least about 40° C. to 65° C.

In some embodiments, the process further comprises (ii) contacting honey with a quantity of chondroitin and glucosamine for a time and under conditions sufficient to form a final concentration in an amount sufficient to provide a therapeutic dose in an acceptable amount of honey for ingestion on a semi-regular, regular or daily basis.

In some embodiments of the process, honey is contacted with chondroitin in an amount sufficient to form a final concentration in the range of 16 mg to 100 mg chondroitin/g honey, and glucosamine in an amount sufficient to form a final concentration in the range of 25 mg to 150 mg glucosamine/g honey.

In some embodiments, honey and chondroitin and glucosamine are heated to at least about 40° C. to 65° C.

In some embodiments, the process comprises (iii) blending liquid honey and chondroitin and glucos amine for at least 12 hours to at least 17 hours. After the blending step, the composition is stably homogeneous and fails to separate on standing.

In another aspect, the present invention provides a process for preparing a composition of honey comprising chondroitin and glucosamine in a substantially stably homogeneous form, the process comprising (i) obtaining honey including optionally heating same to at least about 40° C. to 65° C.; (ii) contacting honey with a quantity of chondroitin sufficient to form a final concentration in the range of 16 mg to 100 mg chondroitin/g honey, and glucosamine sufficient to form a final concentration in the range of 25 mg to 150 mg glucosamine/g honey; (iii) blending liquid honey and chondroitin and glucosamine for at least 12 hours to at least 17 hours. After the blending step, the composition is stably homogeneous and fails to separate on standing.

As described in the examples, initial attempts to form a stably homogeneous composition were unsuccessful and the problem was only solved by blending honey with chondroitin, or chondroitin and glucos amine, for at least 12 hours to at least 17 hours.

In another embodiment, the present invention provides a substantially stably homogeneous composition comprising honey and chondroitin prepared by the following process: (i) obtaining honey; (ii) contacting honey with a quantity of chondroitin sufficient to form a final concentration in the range of 16 mg to 100 mg chondroitin/g honey; and (iii) blending liquid honey and chondroitin for at least 12 hours to at least 17 hours after which the composition is stably homogeneous and fails to separate on standing.

In some embodiments, honey is heated to at least about 40° C. to 65° C. before or after contacting honey with chondroitin.

In another embodiment, the present invention provides a substantially stably homogeneous composition comprising honey and glucosamine prepared by the following process: (i) obtaining honey; (ii) contacting honey with a quantity of glucosamine sufficient to form a final concentration in the range of 25 mg to 150 mg glucosamine/g honey; and (iii) blending liquid honey and glucosamine for at least 12 hours to at least 17 hours after which the composition is stably homogeneous and fails to separate on standing.

In some embodiments, honey is heated to at least about 40° C. to 65° C. before or after honey is contacted with glucosamine.

In another embodiment, the present invention provides a substantially stably homogeneous composition comprising honey and chondroitin and glucosamine prepared by the following process: (i) obtaining honey; (ii) contacting honey with a quantity of chondroitin sufficient to form a final concentration in the range of 16 mg to 100 mg chondroitin/g honey and a quantity of glucosamine sufficient to form a final concentration of 25 mg to 150 mg glucosamine/g honey; and (iii) blending liquid honey and chondroitin and glucosamine for at least 12 hours to at least 17 hours after which the composition is stably homogeneous and fails to separate on standing.

In some embodiments, honey is heated to at least about 40° C. to 65° C. before or after honey is contacted with glucosamine and/or chondroitin.

Methods for determining the concentration of chondroitin and/or glucosamine and for determining the stability and homogeneity of the subject composition are described herein or known to those of skill in the art.

It is to be noted that no exogenous emulsifiers, stabilizers, gelling agents, preservatives, diluents or other modifiers are added to the composition in order to facilitate blending or otherwise to create the subject stably homogeneous compositions.

The above summary is not and should not be seen in any way as an exhaustive recitation of all embodiments of the present invention.

### DETAILED DESCRIPTION

The subject invention is not limited to particular formulations of agents or various medical methodologies, as such may vary.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

By “homogeneous” it is meant that the chondroitin or chondroitin and glucosamine are uniformly distributed throughout the composition.

By “stably homogeneous” it is meant that the composition remains homogeneous, i.e. that the components do not visibly separate or do not become substantially less uniformly distributed during storage. The phrase “substantially less uniformly distributed” includes more than 1% separation. Thus, the stably homogeneous composition of the present invention exhibit less than 1% separation.

By “honey” it is meant including any form of honey from any floral source or blend. Honey has a low water activity which contributes to its anti-microbial activity. The term “honey” means honey that is essentially undiluted. Some dilution may take place as a result of introducing the required amount of chondroitin or glucos amine if these are in liquid form.

By “chondroitin” it is meant any art recognized form of chondroitin, particularly chondroitin sulphate as known in the art. Chondroitin may be derived from a naturally occurring source or may be a synthetic form of chondroitin. Chondroitin is typically derived from animal or aquatic sources. Chondroitin sulphate is commonly derived from bovine, molluscs or fish, such as green lipped mussel or shark cartilage. Chondroitin may be in powdered (dry) form or as a concentrated solution.

By “glucosamine” it is meant any art recognized form of glucosamine, such as glucosamine sulphate or glucosamine hydrochloride. Glucosamine is commonly derived from seafood, crab, lobster or shrimp shells. Glucosamine may be in powdered form or as a concentrated solution.

By “derive from” it is meant that the product is directly or indirectly obtained from a particular source.

Reference to “semi-regular” or “regular” includes daily, every other day, three times per week, etc. Reference to “daily” includes once per day, and divided doses such as half the dose twice per day, a third of the dose three times per day, etc.

A “therapeutic dose” includes a therapeutic dose in respect of osteoarthritis which for chondroitin in human subjects is currently 800 to 1000 mg/day and for glucosamine in human subjects is currently 1500 mg/day. Other therapeutic doses are included. Accordingly, in some embodiment, the arthritis is osteoarthritis or rheumatoid arthritis.

In one broad embodiment, the present invention provides a substantially stably homogeneous composition comprising honey and chondroitin.

In one broad embodiment, the present invention provides a substantially stably homogeneous composition of honey and chondroitin.

The composition is suitable for oral administration to a subject (by a subject) for treatment of arthritis. Subjects are typically human subjects although veterinary application and laboratory animals are not excluded.

In some embodiments, the chondroitin is present in the composition at a concentration sufficient to provide a therapeutic dose in an acceptable amount of honey for ingestion.

Typically, 1 to 2 tablespoons (approximately 23 g to 25 g per tablespoon) of honey is a convenient amount of honey for daily ingestion. This amount of the composition may be dispensed from a container comprising multiple 1 to 2 tablespoon aliquots. Alternatively, the subject compositions may be dispensed into approximately 1 to 2 tablespoon sized sealed containers. Thus, in some embodiments, the subject compositions are provided in unit dosage form.

In some embodiments, the composition comprises a final concentration of at least 16 mg to 100 mg chondroitin/g honey.

The compositions of the present invention are suitable for oral administration to a human subject with or without other food items.

In one broad embodiment, the present invention provides a substantially stably homogeneous composition comprising honey and glucosamine.

In one broad embodiment, the present invention provides a substantially stably homogeneous composition of honey and glucosamine.

In some embodiments, the glucosamine is present in the composition at a concentration sufficient to provide a therapeutic dose in an acceptable amount of honey for ingestion.

In some embodiments, the composition comprises a final concentration of at least 25 mg to 150 mg glucosamine/g honey

In some embodiments, the present invention provides a substantially stably homogeneous composition comprising honey and chondroitin and glucosamine.

In some embodiments, the present invention provides a substantially stably homogeneous composition of honey and chondroitin and glucosamine

In particular embodiments, the chondroitin and glucosamine are present in the composition at concentrations sufficient to provide therapeutic doses in an acceptable amount of honey for ingestion.

In some embodiments, the composition comprises a final concentration of at least 16 mg to 100 mg chondroitin/g honey and at least 25 mg to 150 mg glucosamine/g honey.

In another aspect, the present invention provides a process for preparing a composition of honey comprising chondroitin in a substantially stably homogeneous form, the process comprising (i) obtaining honey.

In some embodiments, honey is heated to at least about 40° C. to 65° C. Honey, or honey and chondroitin may be heated to at least about 40° C. to 65° C. before or after the honey is contacted with chondroitin.

In some embodiments, the process further comprises (ii) contacting honey with a quantity of chondroitin sufficient to form a final concentration that provides a therapeutic dose in an acceptable amount of honey for ingestion on a semi-regular, regular or daily basis.

In some embodiments of the process, honey is contacted with chondroitin in an amount sufficient to form a final concentration in the range of 16 mg to 100 mg chondroitin/g honey.

In some embodiments, the process comprises (iii) blending liquid honey and chondroitin for at least at least 12 hours to at least 17 hours.

Importantly, after the blending step, compositions of the present invention are stably homogeneous and fail to separate on standing.

In some embodiments, the present invention provides a process for preparing a composition of honey comprising chondroitin in a substantially stably homogeneous form, the process comprising (i) obtaining honey including optionally heating same to at least about 40° C. to 65° C.; (ii) contacting honey with a quantity of chondroitin sufficient to form a final concentration in the range of 16 mg to 100 mg chondroitin/g honey; (iii) blending liquid honey and chondroitin for at least 12 hours to at least 17 hours after which the composition is stably homogeneous and fails to separate on standing.

In another aspect, the present invention provides a process for preparing honey comprising glucosamine in a substantially stably homogeneous form, the process comprising (i) obtaining honey.

In some embodiments, honey is heated to at least about 40° C. to 65° C.

In some embodiments, the process further comprises (ii) contacting honey with a quantity of glucosamine sufficient to form a final concentration that provides a therapeutic dose in an acceptable amount of honey for ingestion on a semi-regular or regular daily basis.

In some embodiments of the process, honey is contacted with glucos amine in an amount sufficient to form a final concentration in the range of 25 mg to 150 mg glucosamine/g honey.

In some embodiments, the process comprises (iii) blending liquid honey and glucosamine for at least 12 hours to at least 17 hours Importantly, after the blending step composition is stably homogeneous and fails to separate on standing.

In another aspect, the present invention provides a process for preparing honey comprising glucosamine in a substantially stably homogeneous form, the process comprising (i) obtaining honey and optionally heating same to at least about 40° C. to 65° C.; (ii) contacting honey with a quantity of glucos amine sufficient to form a final concentration in the range of 25 mg to 150 mg glucosamine/g honey; (iii) blending liquid honey and glucosamine for at least 12 hours to at least 17 hours after which the composition is stably homogeneous and fails to separate on standing.

In another aspect, the present invention provides a process for preparing a composition of honey comprising chondroitin and glucosamine in a substantially stably homogeneous form, the process comprising (i) obtaining honey.

In some embodiments, honey is heated to at least about 40° C. to 65° C.

In some embodiments, the process further comprises (ii) contacting honey with a quantity of chondroitin and glucosamine sufficient to form final concentrations that provide therapeutic doses in an acceptable amount of honey for ingestion on a semi-regular, regular or daily basis.

In some embodiments of the process, honey is contacted with chondroitin in an amount sufficient to form a final concentration in the range of 16 mg to 100 mg chondroitin/g honey, and glucosamine in an amount sufficient to form a final concentration in the range of 25 mg to 150 mg glucosamine/g honey.

In some embodiments, the process comprises (iii) blending liquid honey and chondroitin and glucosamine for at least 12 hours to at least 17 hours Importantly, after the blending step, the composition is stably homogeneous and fails to separate on standing.

In another aspect, the present invention provides a process for preparing a composition of honey comprising chondroitin and glucosamine in a substantially stably homogeneous form, the process comprising (i) obtaining honey and optionally heating same to at least about 40° C. to 65° C.; (ii) contacting honey with a quantity of chondroitin in an amount sufficient to form a final concentration in the range of 16 mg to 100 mg chondroitin/g honey, and glucosamine sufficient to form a final concentration in the range of 25 mg to 150 mg glucosamine/g honey; (iii) blending liquid honey and chondroitin and glucosamine for at least 12 hours to at least 17 hours after which the composition is stably homogeneous and fails to separate on standing.

In another embodiment, the present invention provides a substantially stably homogeneous composition of honey and chondroitin prepared by the following process: (i) obtaining honey; (ii) contacting honey with a quantity of chondroitin sufficient to form a final concentration in the range of 16 mg to 100 mg chondroitin/g honey; and (iii) blending liquid honey and chondroitin and glucosamine for at least 12 hours to at least 17 hours after which the composition is stably homogeneous and fails to separate on standing.

In another embodiment, the present invention provides a substantially stably homogeneous composition of honey and glucosamine prepared by the following process: (i) obtaining honey; (ii) contacting honey with a quantity of glucosamine sufficient to form a final concentration in the range of 25 mg to 150 mg glucosamine/g honey; and (iii) blending liquid honey and glucosamine for at least 12 hours to at least 17 hours.

In another embodiment, the present invention provides a substantially stably homogeneous composition of honey and chondroitin and glucosamine prepared by the following process: (i) obtaining honey; (ii) contacting honey with a quantity of chondroitin sufficient to form a final concentration in the range of 16 mg to 100 mg chondroitin/g honey and a quantity of glucosamine sufficient to form a final concentration of 25 mg to 150 mg glucosamine/g honey; and (iii) blending liquid honey and chondroitin and glucosamine for at least 12 hours to at least 17 hours.

In another embodiment, the present invention provides a process for preparing a composition comprising honey and chondroitin, or honey and glucosamine, or honey and chondroitin and glucosamine in a stably homogeneous form, the process comprising: (i) obtaining honey; (ii) contacting honey with a quantity of chondroitin sufficient to provide a final concentration in the range of 16 mg to 100 mg chondroitin/g honey and/or a quantity of glucosamine sufficient to provide a final concentration of 25 mg to 150 mg glucosamine/g honey; and (iii) blending liquid honey and chondroitin, or honey and glucos amine, or honey and chondroitin and glucos amine for at least 12 hours to at least 17 hours after which the composition is stably homogeneous and fails to separate on standing.

In another embodiment, the present invention provides a stably homogeneous composition of honey and chondroitin, or honey and glucosamine, or honey and chondroitin and glucosamine prepared by the following process: (i) obtaining honey; (ii) contacting honey with a quantity of chondroitin sufficient to provide a final concentration in the range of 16 mg to 100 mg chondroitin/g honey and/or a quantity of glucos amine sufficient to provide a final concentration of 25 mg to 150 mg glucosamine/g honey; and (iii) blending liquid honey and chondroitin, or honey and glucos amine, or honey and chondroitin and glucos amine for at least 12 hours to at least 17 hours after which the composition is stably homogeneous and fails to separate on standing.

In another embodiment, the present invention provides a process as herein described or the composition as herein described including heating honey to at least about 40° C. to 60° C. before or after addition of chondroitin and/or glucosamine.

Methods for determining the concentration of chondroitin and/or glucosamine and for determining the activity, stability and homogeneity of the subject composition are described herein or known to those of skill in the art.

It is to be noted that no exogenous emulsifiers, stabilizers, gelling agents, preservatives, diluents or other modifiers are added to the composition in order to facilitate blending or otherwise to create the subject stably homogeneous compositions.

Prior to the present invention, it was not possible to develop a honey product with the required amounts of chondroitin sulphate and glucosamine to provide recommended therapeutic quantities of chondroitin in an acceptable volume of honey for routine (such as daily) ingestion. It was not known, for example, how hot the honey would have to be heated in order to dissolve chondroitin sulphate or how to achieve effective blending and whether the integrity of the honey or the biological activity of the active ingredients would be reduced thereby.

Reference to 16 mg to 100 mg chondroitin/g honey includes 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg or 100 mg chondroitin/g honey and any amounts in between these values.

Reference to 25 mg to 150 mg glucosamine/g honey includes 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg or 150 mg glucosamine/g honey, and any amounts in between these values.

Reference to at least about 40° C. to 65° C. includes 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C. or 65° C. and any temperature in between these values.

Reference to 12 hours to 24 hours includes 12 hours, 13 hours, 14 hours, 15 hours, 16 hours, 17 hours, 18 hours, 19 hours, 20 hours, 21 hours, 22 hours, 23 hours, or 24 hours and any times in between these times.

Honey does not naturally contain glucosamine or chondroitin sulphate in significant amounts or at levels that would aid arthritis.

In some embodiments, the composition is prepared by using at least 70% to 95% by final volume of undiluted honey. Reference to 70% to 95% includes preparations prepared using 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% of final volume of undiluted honey.

In some embodiments, the composition comprises approximately 5% to 20% by volume of a solution of 98% pure chondroitin sulphate.

Reference to 5% to 20% includes 5%, 6%, 7%, 8%, 9%, 10%, 11%, 12%, 13%, 14%, 15%, 16%, 17%, 18%, 19% or 20% by volume of a solution of 98% pure chondroitin sulphate.

In some embodiments, the chondroitin sulphate is liquid bovine chondroitin sulphate. In one illustrative embodiment, the liquid preparation is at least 98% pure bovine chondroitin sulphate.

As described in the Examples, it was surprisingly possible to produce a honey preparation comprising therapeutically acceptable dosages of chondroitin, or chondroitin and glucosamine, in an acceptable volume of honey for daily ingestion by blending the components for at least 12 hours to at least 17 hours.

The amount of the subject composition to provide a therapeutic dose is generally between 1 to 2 tablespoons per day (1 tablespoon honey is 23 g to 25 g).

Honey is generally filled into sealable containers which may be coloured or otherwise constructed to avoid or reduce contact between the composition and light.

The present invention is further described by the following non-limiting Examples.

### EXAMPLES

### Example 1

### Methodology for Producing the Subject Composition

The blending of the product is highly technical and complex in nature.

The functionality and technical viability of ArthriHoney® has been determined by preparing the honey in such a way as to ensure that the integrity of the honey is not damaged through excessive processing temperatures and that the required concentrations of chondroitin sulphate and glucos amine are achieved in the final product. These objectives have been supported through trials that:
(i) confirmed theoretical calculations of the pre-processing concentrations of chondroitin sulphate and glucosamine to achieve the required product concentration through measured final compositions; and
(ii) demonstrated the maintenance of temperatures below the critical values that affect the processing integrity of honey.

Honey is usually heated up to at least about 40° C. to 65° C. to make it less viscous when packaging. In some cases, honey is heated to 65° C. to “pasteurize” the honey.

In the present process, liquid honey is blended with chondroitin and glucosamine on a ratio basis depending on the final dose required. The mixture is blended in a mixer over a period of time so that the product remains homogeneously mixed during standing. Initial trials were conducted with a domestic food processor at low speed for various times.

If correctly blended, the honey acts as a natural preservative and the stability of the product has been achieved. The shelf life can be confirmed with accelerated testing. Thus far, compositions have been stored for 2 years and no visible (less than 1%) separation has taken place.

Various varieties of honey were tested in trials with no significant variations observed. The main criteria for the honey is taste.

The final product is tested for chondroitin or chondroitin and glucosamine concentrations at an independent clinical laboratory. Various methods can be used to test for levels of chondroitin and evaluation of loss of activity over time. In an illustrative example, high performance liquid chromatography after solid phase extraction and degradation of chondroitin polymer to disaccharide may be employed.

The monograph or compendial standard with which active constituents are required to comply is British Pharmacopoeia (BP); or European Pharmacopoeia (EP); or United States Pharmacopoeia (USP) for chondroitin sulphate and United States Pharmacopoeia (USP) for glucosamine sulphate or glucosamine hydrochloride.

In some embodiments, pure liquid chondroitin from bovine cartilage is employed. 5% to 15% by volume 98% pure liquid bovine cartilage extracts were added to honey with successful blending outcomes.

In initial trials, honey was heated to liquid and then chondroitin or chondroitin and glucosamine were blended with honey for varying periods of time with no success. Specifically, initially different concentrations of chondroitin sulphate were mixed with honey for one hour, four hours, twelve hours and sixteen hours with no success.

Subsequently, blending trials were conducted with blending for more than 24 hours and, surprisingly, it was found that a stable homogeneous composition could be obtained. Specifically, chondroitin or chondroitin and glucosamine each at 35 mg to 90 mg per millilitre honey were dissolved in honey and blended for 24 hours. The blending time was then gradually reduced and successful blending was also determined to be achieved at 19 hours and then 17 hours and 12 hours blending depending upon various parameters such as how liquid the honey is or the temperature or blender used.

Different concentrations of chondroitin or chondroitin and glucosamine were then blended with honey for more than 17 hours to investigate the concentration of chondroitin or chondroitin and glucosamine that can be successfully homogenized. Specifically, a solution of 98% pure bovine chondroitin was blended with honey at 5%, 10%, 15% and 20% (i.e., 1/20, 1/10, 1/6.67, 1/5, respective dilutions). The 5%, 10% and 15% mixtures blended successfully while the 20% blend presented a milky, opaque appearance indicating that the mixture would not remain homogeneous.

The results of these initial trials indicate that homogeneous concentrations of 16 mg to 100 mg chondroitin/g honey are achievable which are suitable for providing a therapeutic dose of 800 to 1000 mg per day chondroitin sulphate in 25 g to 50 g honey. Similarly, for glucosamine, concentrations of 25 mg to 150 mg glucosamine/g honey are achieved that will provide a therapeutic dosage of 1500 mg per day in 25 to 50 g honey.

“Treatment” includes “prophylaxis”. These terms are used in their broadest context and include any measureable or statistically significant amelioration in at least some subjects in one or more symptoms of arthritis or in the risk of developing or rate of onset of more severe symptoms of arthritis.

Many modifications will be apparent to those skilled in the art without departing from the scope of the present invention.

What is claimed is:

1. A stable homogeneous composition comprising honey and chondroitin suitable for oral administration for treatment of arthritis.

2. The composition of claim 1 further comprising glucosamine.

3. The composition of claim 1 wherein the composition comprises a therapeutic dose of chondroitin suitable for the oral treatment of arthritis.

4. The composition of claim 1 wherein the composition comprises a therapeutic dose of glucosamine suitable for the oral treatment of arthritis.

5. The composition of claim 1 wherein the composition comprises a final concentration of 16 mg to 100 mg chondroitin/g honey.

6. The composition of claim 1 wherein the composition comprises a final concentration of 25 mg to 150 mg glucosamine/g honey.

7. A process for preparing a composition comprising honey and chondroitin in a stable homogeneous form, the process comprising
(i) obtaining honey;
(ii) contacting honey with a quantity of chondroitin sufficient to form a final concentration in the range of 16 mg to 100 mg chondroitin/g honey;
(iii) blending liquid honey and chondroitin for at least 12 hours to at least 17 hours after which the composition is stably homogeneous and fails to separate on standing.

8. The process of claim 7, wherein step (ii) further comprises contacting said honey with a quantity of glucosamine sufficient to form a final concentration in the range of 25 mg to 150 mg glucosamine/g honey, and wherein step (iii) comprises blending said liquid honey and chondroitin and glucosamine for at least 12 hours to at least 17 hours after which the composition is stably homogeneous and fails to separate on standing.

9. The process of claim 7 including heating honey to at least about 40° C. to 60° C. before or after addition of chondroitin and/or glucosamine.

10. A stable homogeneous composition of honey and chondroitin prepared by the following process:
(i) obtaining honey;
(ii) contacting honey with a quantity of chondroitin sufficient to provide a final concentration in the range of 16 mg to 100 mg chondroitin/g honey; and
(iii) blending liquid honey and chondroitin for at least 12 hours to at least 17 hours after which the composition is stably homogeneous and fails to separate on standing.

11. The composition of claim 10, wherein step (ii) further comprises contacting said honey with a quantity of glucos amine sufficient to form a final concentration in the range of 25 mg to 150 mg glucosamine/g honey, and wherein step (iii) comprises blending said liquid honey and chondroitin and glucosamine for at least 12 hours to at least 17 hours after which the composition is stably homogeneous and fails to separate on standing.

12. The composition of claim 10 including heating honey to at least about 40° C. to 60° C. before or after addition of chondroitin and/or glucosamine.
